(19) United States
(12) Patent Application Publication
(10) Pub. No.: US 2015/0216791 A1
(43) Pub. Date: Aug. 6, 2015

(54) ESSENTIAL OIL OF SWEET MARJORAM, COMPOSITION COMPRISING IT, COSMETIC TREATMENT METHOD AND USE

(71) Applicant: L'OREAL, Paris (FR)

(72) Inventors: Geraldine Lerebour, Les Loges (FR); Pierre Lartaud, Eurre (FR); Agnes Pegeon, Le Plessis Robinson (FR)

(21) Appl. No.: 14/409,077

(22) PCT Filed: Jun. 28, 2013
(86) PCT No.: PCT/EP2013/063635

§ 371 Date: Dec. 18, 2014

Related U.S. Application Data
(60) Provisional application No. 61/695,320, filed on Aug. 31, 2012.

(30) Foreign Application Priority Data
Jul. 2, 2012 (FR) 1256312

(51) Int. Cl. A61K 8/92; A61Q 5/02; A61Q 5/00; A61K 8/34; A61K 8/31

(52) U.S. Cl. CPC A61K 8/922; A61K 8/34; A61K 8/347; A61K 8/31; A61Q 5/006; A61Q 5/02

(57) ABSTRACT

The present invention relates to a novel essential oil of sweet marjoram, to the cosmetic or pharmaceutical compositions comprising it, and also to a cosmetic treatment method employing it and to its cosmetic use as anti-dandruff agent.

The present invention relates to the cosmetic use of a specific essential oil of sweet marjoram as anti-dandruff agent, in particular in the cosmetic treatment of aesthetic disorders of the scalp, related in particular to dandruff conditions. The invention also relates to a cosmetic treatment method intended to remove and/or reduce dandruff, in particular that caused by yeasts of the genus Malassezia, employing the said essential oil, and also to a composition, in particular a cosmetic composition, comprising the said essential oil, and to the said essential oil as such.

Dandruff problems affect up to 50% of the world's population. They affect both men and women and are perceived as having a very negative psychosocial impact. The appearance of dandruff is disagreeable both aesthetically and because of the annoyance which it causes (in particular itching), so that many people confronted with this problem to variable degrees wish to be rid of it efficiently and permanently.

Dandruff corresponds to excessive and visible desquamation of the scalp resulting from the excessively rapid multiplication of the epidermal cells and from their abnormal maturation. This phenomenon can be brought about in particular by excessively aggressive hair treatments, extreme climatic conditions, nervousness, diet, fatigue or pollution.

Dandruff conditions generally result from a disorder of the microflora of the scalp and more particularly from the excessive colonization by a fungus which belongs to the family of the yeasts of the genus Malassezia and which is naturally present on the scalp.

Many anti-dandruff treatments have been developed with the main objective of eradicating Malassezia yeasts from the scalp. Thus, the activity of the antidandruff active agents of today, such as zinc pyrithione, piroctone olamine or selenium disulfide, is based mainly on their fungicidal property. However, these antidandruff agents are not completely satisfactory in terms of effectiveness (immediate effectiveness or duration of the effect) and/or in terms of impact on the environment.

The aim of the present invention is to provide a cosmetic anti-dandruff agent which does not irritate the skin and scalp and which is more effective than known antidandruff agents, while having a more favourable impact in terms of the environment (low bioaccumulation and good biodegradability, in particular); the said cosmetic active agent making it possible in particular to re-establish the ecoflora of the scalp and in particular to prevent excessive colonization of the scalp by Malassezia sp.

The Applicant Company has discovered, surprisingly, that the use of a specific essential oil of sweet marjoram makes it possible to effectively treat aesthetic disorders of the scalp related to a dandruff condition, in particular those associated with the proliferation of yeasts of the genus Malassezia, and to overcome the disadvantages of the prior art.

It has been observed that, by employing the essential oil according to the invention, it is possible to remove and/or reduce the amount of dandruff present on the hair and/or scalp, and the generally associated itching of the scalp.

It has also been observed that the use of the said essential oil makes it possible in addition to improve the quality of the keratinous fibre (the individual hair) and to contribute cosmetic properties thereto, in particular of conditioning and very particularly of smoothing, suppleness, sheen, disentangling, softness, manageability and volume. In addition, the conditions of use of the composition are also improved, in particular the distribution of the composition over the fibre, and also the rinsing thereof, for example with water.

A subject-matter of the present invention is thus an essential oil of Origanum majorana and/or of its hybrids with thuyanol, characterized in that it comprises at least 18% by weight, preferably at least 20% by weight, of cis-4-thujanol and at least 16% by weight, preferably at least 18% by weight, indeed even at least 20% by weight, of carvacrol.

The invention also relates to a cosmetic or pharmaceutical composition comprising at least the said essential oil of Origanum majorana.

The invention further relates to a cosmetic treatment method intended to treat aesthetic disorders of the scalp related to a dandruff condition and/or to remove and/or reduce dandruff, characterized in that it comprises the application, to the hair and/or scalp, of the said essential oil or of a cosmetic composition comprising it.

The invention further relates to the cosmetic use of the said essential oil or of a cosmetic composition comprising it as anti-dandruff agent or also for the treatment of aesthetic disorders of the scalp related to a dandruff condition.

In the present invention, the term “keratinous substance” is understood in particular to mean the skin (face, body, scalp), hair, eyelashes, eyebrows and nails.

Within the meaning of the present invention, the term “physiologically acceptable medium” is understood to mean a medium which is suitable for the topical administration of a composition. Preferably, it is a cosmetically acceptable medium, that is to say without an unpleasant odour and/or unpleasant appearance.

Essential oils are products obtained from starting materials of plant origin (leaves, stems, flowers or whole plant, for example).

These essential oils can be obtained according to different processes, such as steam distillation, distillation or extraction by means of volatile solvents, in particular.

According to the definition given in the international standard ISO 9235 and adopted by the Commission of the European Pharmacopoeia, an essential oil is an odorous product, generally of complex composition, obtained from a botanically defined plant starting material, either by steam distillation, or by dry distillation, or by an appropriate mechanical process without heating (cold expression). The essential oil is generally separated from the aqueous phase by a physical process which does not result in any significant change in the composition.

The choice of the technique for obtaining an essential oil depends mainly on the starting material: its original state and its characteristics, its nature proper. The “essential oil/plant starting material” yield can be extremely variable depending on the plants: from 15 ppm to more than 20%. This choice conditions the characteristics of the essential oil, in particular viscosity, colour, solubility, volatility, and richness or poorness in certain constituents.

Mention may be made, among the methods for obtaining an essential oil, of steam distillation, which can, for example, be carried out by dry distillation or hydrodistillation.

Hydrodistillation can be carried out on a glass apparatus, such as that defined in the European Pharmacopoeia for the determination of the essential oil from a plant material.

Steam distillation corresponds to the vaporization, in the presence of steam, of a substance which is not very miscible with water The starting material is brought together with water brought to boiling point (hydrodistillation) or with steam in a still (dry distillation). The steam entrains the essential oil vapour, which is condensed in the condenser in order to be recovered as liquid phase in a Florentine flask (or essence jar), where the essential oil is separated from the water by settling. The term “aromatic water” or “hydrolat” or “distilled floral water” is used to describe the aqueous distillate which remains after the steam distillation, once the essential oil has been separated.

Essential oils are generally volatile and liquid at ambient temperature (25° C.), which distinguishes them from “fixed” oils. They are more or less coloured and their density is generally less than that of water. They have a high refractive index and most of them deflect polarized light. They are liposoluble and soluble in the usual organic solvents, steam distillable and very sparingly soluble in water.

The plant Origanum majorana is a plant of the Lamiaceae family. It is an annual or perennial plant having a height of approximately 50 to 60 cm. This plant can in particular be found in the south of France. It is more commonly known under the name of sweet marjoram.

According to the invention, the essential oil capable of being used is an essential oil of Origanum majorana and/or an essential oil of the hybrids with thuyanol; preferably, the essential oil of Origanum majorana is used.

Advantageously, the essential oil capable of being used according to the present invention is an essential oil of Origanum majorana L., in particular of Origanum majorana CT thujanol, also known as Origanum x majoricum Cambassedes.

The essential oil of Origanum majorana in accordance with the invention can advantageously be obtained from the aerial part of the plant. Furthermore, harvesting can be carried out at different stages of cutting: beginning of flowering or end of flowering and preferably at the end-of-flowering stage.

The chemical composition of the essential oil of Origanum majorana in accordance with the invention can be analysed by conventional techniques known to a person skilled in the art, such as gas chromatography GC analysis, chromatographic analysis with flame ionization detection, referred to as GC-FID, or GC/MS analysis, which consists of the use of a mass spectrometer coupled to a gas chromatograph.

The essential oil of Origanum majorana and/or of its hybrids with thuyanol in accordance with the invention is characterized in that it comprises at least 18% by weight of cis-4-thujanol and at least 16% by weight of carvacrol.

The essential oils of Origanum majorana are generally known in the literature; however, none of them comprises these two components in these specific contents.

The two predominant components participating in the composition of the essential oil in accordance with the invention are thus:
cis-4-thujanol, present at a content of greater than or equal to 18% by weight, preferably of greater than or equal to 20% by weight, and in particular at a content ranging from 18% to 70% by weight, especially from 20% to 50% by weight and preferably from 22% to 35% by weight, with respect to the total weight of the components of the essential oil;
carvacrol, present at a content of greater than or equal to 16% by weight, in particular of greater than or equal to 18% by weight, preferably of greater than or equal to 20% by weight, and in particular at a content ranging from 16% to 70% by weight, especially from 18% to 50% by weight, better still from 20% to 40% by weight and preferably from 22% to 25% by weight, with respect to the total weight of the components of the oil.

Preferably, the following components are also generally present, in particular each at a concentration generally of greater than 1.8% by weight, with respect to the total weight of the components, especially ranging from 1.8% to 10% by weight, indeed even from 2% to 8% by weight:
terpinen-4-ol,
γ-terpinene,
para-cymene,
sabinene,
α-terpinene,
trans-4-thujanol.

The essential oil of Origanum majorana and/or of the hybrids in accordance with the invention can be present in the composition at a content of between 0.001% and 5% by weight, especially between 0.01% and 3% by weight, more particularly between 0.05% and 2% by weight and better still between 0.1% and 1% by weight, with respect to the total weight the composition.

The cosmetic compositions capable of being used in the context of the invention generally comprise a cosmetically acceptable medium, that is to say a medium compatible with keratinous substances, such as the skin of the face or body, the hair, the eyelashes, the eyebrows and the nails.

The cosmetic composition used according to the invention can be a composition which can be rinsed-out or left-in. The said composition, in particular hair composition, is preferably a shampoo, a cream, a foam (aerosol or non-aerosol), a paste, a gel, an emulsion, a lotion or a stick. Preferably, the cosmetic composition is provided in the form of a shampoo, of a gel or of a lotion, in particular a hair one, preferably an anti-dandruff one.

The cosmetic composition can be anhydrous or can comprise, preferably, an aqueous or aqueous/organic medium; it can thus comprise water and/or one or more organic solvents which can be chosen from linear or branched C₁-C₆ monoalcohols, such as ethanol, isopropanol, tert-butanol or n-butanol; polyols, such as glycerol, polypropylene glycol, hexylene glycol (or 2-methyl-2,4pentanediol) and polyethylene glycols; polyol ethers, such as dipropylene glycol monomethyl ether; and their mixtures.

Preferably, the cosmetic composition comprises an amount of organic solvents ranging from 0.05% to 60% by weight, preferably from 0.5% to 50% by weight and better still from 1% to 40% by weight, with respect to the total weight of the cosmetic composition.

In addition, the cosmetic composition can advantageously comprise at least one additional constituent normal in cosmetics, such as, in particular, thickeners; surfactants chosen from anionic, cationic, non-ionic, amphoteric and/or zwitterionic surfactants; conditioning agents; silicones; agents for combating hair loss; other anti-dandruff agents; vitamins; waxes; sunscreens; coloured or colourless inorganic or organic pigments; dyes; pearlescent and opacifying agents; sequestering agents; plasticizing agents; fragrances; or preservatives. Of course, a person skilled in the art will take care to choose this or these optional additional compounds and/or their amounts in such a way that the advantageous properties of the composition according to the invention are not, or not substantially, detrimentally affected by the envisioned addition.

According to one embodiment, the cosmetic composition can be an aqueous composition. The term “aqueous” is understood to mean a composition for which the content of free water is greater than 10% by weight, preferably greater than 30% by weight, better still greater than 50% by weight and indeed even greater than 70% by weight, with respect to the total weight of the composition.

According to another embodiment, the cosmetic composition can be an anhydrous composition. The term “anhydrous” is understood to mean a composition for which the content of free or added water is less than 10% by weight, in particular less than 3% by weight, and preferably for which the content of added water is less than 1% by weight, with respect to the total weight of the composition. Preferably, the anhydrous cosmetic composition does not comprise water.

The cosmetic composition may or may not be rinsed off after having been applied to the keratinous substances (hair and/or scalp).

Furthermore, after the application of the cosmetic composition according to the invention, it is possible to apply, to the surface of the keratinous substances, a composition comprising one or more active agents chosen from antibacterials, antifungals and/or powders.

A further subject-matter of the invention is a cosmetic treatment method intended to remove and/or reduce dandruff, in particular that brought about by yeasts of the genus Malassezia, characterized in that it comprises the application, to the hair and/or scalp, of at least one essential oil of Origanum majorana and/or of the hybrids with thuyanol as defined in accordance with the invention or of a cosmetic composition comprising it.

The application of the said oil or of the composition comprising it may or may not be followed by a stage of rinsing with water. Preferably, this cosmetic treatment method is repeated at the rate of at least twice weekly.

It has been found that the said method makes it possible in particular to effectively treat aesthetic disorders of the scalp related to a dandruff condition.

The invention is illustrated in greater detail in the following examples.

### EXAMPLE 1

An essential oil of Origanum majorana was prepared by distillation of 300 g of fresh aerial part, collected at the blossomed to deblossomed stage, in an apparatus of Clevenger type, by dry distillation for 1 h 30 min. An essential oil was obtained with a yield of the order of 1.05%.

The essential oil thus obtained comprises:

| | |
|---|---|
| terpinen-4-ol | 9.2% |
| γ-terpinene | 7.4% |
| p-cymene | 2.2% |
| sabinene | 5.3% |
| α-terpinene | 3.8% |
| trans-4-thujanol | 2.8% |
| cis-4-thujanol | 25.6% |
| α-terpineol | 2.3% |
| linalyl acetate | 3.0% |
| carvacrol | 20.4% |

The composition of the essential oil obtained was determined by GC (gas chromatography) and mass spectrometry.

### EXAMPLE 2

### Principle:

By a method of dilution in a liquid medium, bringing different concentrations of product into contact with a nutritive broth inoculated with the test strain. After incubation, counting the surviving microorganisms (log).

### Protocol:

bringing the product into contact, at a concentration double the test concentration, with a doubly concentrated nutritive broth assaying approximately between 2 and 6.10⁵ CFU/ml.
after incubating at 32.5° C.±2.5° C. for 24 hours, the surviving microorganisms are counted by spiral inoculation and compared with the starting inoculum in order to define the degrees of reduction obtained.
preparation of the product subjected to the test: a 10% mother solution is prepared in 1% agar.
after stirring, dilutions are carried out in order to prepare 0.02%, 0.1%, 0.2%, 1% and 2% solutions.

Spiral inoculation systems use a semi-automatic inoculater of the AES® or Interscience® type, which deposits a calibrated volume of a liquid sample at the surface of an agar placed on a rotating plate, while describing an Archimedean spiral. After incubation, reading is carried out using graphs. This technique makes it possible to carry out the bacterial count of a sample on one and the same dish, dispensing with all or some of the intermediate dilutions. This methodology is much used and is an officially accepted technique.

### Operating Conditions:

concentrations of test product: 0.01%, 0.05%, 0.1%, 0.5% and 1% (v/v or w/v)
diluant used: 1% agar
appearance in the broth: opaque emulsion

### Results:

After having inoculated 6.7 log in the medium comprising different concentrations of the essential oil, complete decontamination of the microbial population is observed after 24 hours at 1%; the population is decreased by 3.6 log at 0.5%.

| | Pityrosporum ovale | | | | |
|---|---|---|---|---|---|
| | 0.01% | 0.05% | 0.1% | 0.5% | 1% |
| Essential oil of Origanum | 6.1 | 5.4 | 5.4 | 3.1 | 0 |
| majorana | | | | | |
| (Example 1) | | | | | |

### EXAMPLE 3

An anti-dandruff shampoo is prepared, comprising (% by weight):

| | |
|---|---|
| Sodium Laureth Sulfate | 12% |
| Disodium Cocoamphodiacetate | 3.5% |
| Essential oil of Origanum majorana (Example 1) | 0.5% |
| Glycol Distearate | 2% |
| Cocamide MIPA (and) Isopropanolamine | 1.5% |
| Salicylic Acid | 0.5% |
| Piroctone Olamine | 0.5% |
| HCl | q.s. pH 7 |
| Water | q.s. for 100% |

1. Essential oil of Origanum majorana and/or of its hybrids with thuyanol, characterized in that it comprises at least 18% by weight, of cis-4-thujanol and at least 16% by weight of carvacrol.

2. Essential oil according to claim 1, which is an essential oil of Origanum majorana L.

3. Essential oil according to claim 1, comprising:
cis-4-thujanol at a content ranging from 18% to 70% by weight with respect to the total weight of the components of the essential oil; and
carvacrol at a content ranging from 16% to 70% by weight with respect to the total weight of the components of the oil.

4. Essential oil according to claim 1, additionally comprising the following components, each present at a concentration of greater than 1.8% by weight, with respect to the total weight of the components:
terpinen-4-ol,
γ-terpinene,
para-cymene,
sabinene,
α-terpinene,
trans-4-thujanol.

5. Cosmetic or pharmaceutical composition comprising at least one essential oil of Origanum majorana as defined in claim 1.

6. Composition according to claim 5, comprising the said essential oil at a content of between 0.001% and 5% by weight with respect to the total weight of the composition.

7. Composition according to claim 5, comprising at least one additional constituent chosen from thickeners; anionic, cationic, non-ionic, amphoteric and/or zwitterionic surfactants; conditioning agents; silicones; agents for combating hair loss; other anti-dandruff agents; vitamins; waxes; sunscreens; coloured or colourless inorganic or organic pigments; dyes; pearlescent and opacifying agents; sequestering agents; plasticizing agents; fragrances; or preservatives.

8. Composition according to claim 5, which is provided in the form of a shampoo, of a gel or of a lotion.

9. Cosmetic treatment method intended to treat aesthetic disorders of the scalp related to a dandruff condition and/or to remove and/or reduce dandruff, which comprises the application, to the hair and/or scalp, of an essential oil as defined in claim 1 or of a cosmetic composition thereof.

10. Method according to claim 9, in which the application of the said oil or of the said composition is followed by a stage of rinsing with water.

11. Cosmetic use of an essential oil as defined in claim 1 or of a cosmetic composition thereof as anti-dandruff agent.

12. Cosmetic use of an essential oil as defined in claim 1 or of a cosmetic composition thereof for treating aesthetic disorders of the scalp related to a dandruff condition.

13. Essential oil according to claim 2, comprising:
cis-4-thujanol at a content ranging from 18% to 70% by weight with respect to the total weight of the components of the essential oil; and
carvacrol at a content ranging from 16% to 70% by weight with respect to the total weight of the components of the oil.

14. Essential oil according to claim 2, additionally comprising the following components, each present at a concentration of greater than 1.8% by weight, with respect to the total weight of the components:
terpinen-4-ol,
γ-terpinene,
para-cymene,
sabinene,
α-terpinene,
trans-4-thujanol.

15. Essential oil according to claim 3, additionally comprising the following components, each present at a concentration of greater than 1.8% by weight, with respect to the total weight of the components:
terpinen-4-ol,
γ-terpinene,
para-cymene,
sabinene,
α-terpinene,
trans-4-thujanol.

16. Cosmetic or pharmaceutical composition comprising at least one essential oil of Origanum majorana as defined in claim 2.

17. Cosmetic or pharmaceutical composition comprising at least one essential oil of Origanum majorana as defined in claim 3.

18. Cosmetic or pharmaceutical composition comprising at least one essential oil of Origanum majorana as defined in claim 4.

19. Composition according to claim 6, comprising at least one additional constituent chosen from thickeners; anionic, cationic, non-ionic, amphoteric and/or zwitterionic surfactants; conditioning agents; silicones; agents for combating hair loss; other anti-dandruff agents; vitamins; waxes; sunscreens; coloured or colourless inorganic or organic pigments; dyes; pearlescent and opacifying agents; sequestering agents; plasticizing agents; fragrances; or preservatives.

20. Composition according to claim 6, which is provided in the form of a shampoo, of a gel or of a lotion.
